(19) United States
(12) Patent Application Publication
(10) Pub. No.: US 2023/0241322 A1
(43) Pub. Date: Aug. 3, 2023

(54) Improvements of an optical sensing system of a drug delivery device

(71) Applicant: Sanofi, Paris (FR)

(72) Inventors: Craig Ashley Mason, Helsby Cheshire (GB); Oliver Charles Gazeley, Basel (CH); Aidan Michael O`Hare, Warwick Warwickshire (GB); Matthew Francis Hobson, Warwick Warwickshire (GB); Alexander Allerdings, Frankfurt am Main (DE)

(21) Appl. No.: 18/011,773

(22) PCT Filed: Jun. 25, 2021
(86) PCT No.: PCT/EP2021/067443

§ 371 Date: Dec. 20, 2022

(30) Foreign Application Priority Data
Jun. 26, 2020 (EP) 20315325.9

(51) Int. Cl. A61M 5/315; G01D 5/347

(52) U.S. Cl. CPC A61M 5/31551; A61M 5/31568; G01D 5/34723; A61M 2205/50; A61M 2205/3306; A61M 2205/3317

(57) ABSTRACT

An optical guiding device configured for application with a drug delivery device is described. The drug delivery device has (i) a movable dosage programming component including a rotary encoder system and (ii) a sensor arrangement including at least one optical sensor. The at least one optical sensor is configured to detect movement of the movable dosage programming component relative to the sensor arrangement during dosing of a drug by emitting radiation and detecting at least one reflections the emitted radiation from the rotary encoder system. The optical guiding device is configured for guiding the radiation and the at least one reflection of the emitted radiation and includes at least one light pipe having the shape of a frustum with a sensor-side surface and a encoder-side surface.

### CROSS REFERENCE TO RELATED APPLICATIONS

The present application is the national stage entry of International Patent Application No. PCT/EP2021/067443, filed on Jun. 25, 2021, and claims priority to Application No. EP 20315325.9, filed on Jun. 26, 2020, the disclosures of which are incorporated herein by reference.

### TECHNICAL FIELD

The present disclosure relates to improvements of an optical sensing system of a drug delivery device.

### BACKGROUND

WO 2019/101962 A1 discloses a module configured to be used with or applied to a medicament injection device comprising a movable dosage programming component with a rotary encoder system, particularly an injection device as described herein, the module comprising: a sensor arrangement comprising at least one optical sensor being configured to detect movement of the movable dosage programming component of the injection device relative to the sensor arrangement during dosing of a medicament and a collimating optics being arranged between the at least one optical sensor and the movable dosage programming component; and a processor arrangement configured to, based on said detected movement, determine a medicament dosage administered by the injection device. The collimating optics may comprise one or more of the following: one or more discrete collimating lenses; one or more light pipes. A discrete collimating lens may be arranged between each optical sensor and each light pipe and/or between each light pipe and the movable dosage programming component. A single discrete collimating lens may be provided for each sensor and configured to cover the transmitter and/or receiver portion of the sensor. The single discrete lens may be a lens array covering the sensor, particularly a micro-moulded lens array. The one or more light pipes may have the shape of a frustum, particularly with a circular or an elliptic base.

### SUMMARY

This disclosure describes different aspects of improvements of an optical sensing system of a drug delivery device. The optical sensing system may comprise a sensor arrangement with at least one optical sensor and a movable dosage programming component comprising a rotary encoder system. The at least one optical sensor is configured to detect movement of the movable dosage programming component relative to the sensor arrangement during dosing of a drug by emitting radiation and detecting reflections of the emitted radiation from the rotary encoder system. Optical guiding means are provided, which are configured for guiding the radiation emitted by the at least one optical sensor to the geometrically separated rotary encoder system and for guiding reflections of the emitted radiation from the rotary encoder system back to the at least one optical sensor. Such an optical sensing system is for example disclosed in WO 2019/101962 A1, FIGS. 21 to 29 and FIG. 36 with the corresponding description.

In one aspect the present disclosure provides optical guiding means comprising at least one light pipe having the shape of a frustum with a sensor-side surface and a encoder-side surface, wherein the at least one light pipe comprises one or more of the following features:

the ratio of the sensor-side surface and the encoder-side surface being equal to or larger than about 1.0;
the ratio of the surface of a discreet encoding target of the rotary encoder system and the encoder-side surface being equal to or larger than 1.0;
the encoder-side surface having a roughness with an average feature size in the range of the wavelength of the radiation emitted by the at least one optical sensor;
the sensor-side surface forms a single lens face being moulded as part of the optical guiding means, wherein the single lens face is formed as collimating optics.

The ratio of surfaces as described herein particularly relates to the ratio of the size or area of the surfaces. These features may improve the guiding of the radiation by the at least one light pipe such that a better detection signal may be generated by the at least one optical sensor. Particularly, the specific geometric relationships and the application of the specific surface finish of the encoder-side surface may improve the radiation guiding by a light pipe since this may incur a more focused reception of reflected radiation and, thus, result in a larger signal amplitude of the at least one optical sensor producing an output signal for further processing. Particularly, the waveform of the signal generated by the at least one optical sensor when the movable dosage programming component with the rotary encoder system is rotated for example for dosage selection may comprise larger swings and common rail voltages compared to a signal generated with an optical sensing system without these optical guiding means. This allows more easily and reliably encoding the generated signal by a processor and to more reliably detecting a selected dosage. The moulding of a signal lens face as part of the optical guiding means may reduce the complexity of the optical guiding means since fewer parts are required. Furthermore, the production of the optical guiding means may be made less complex since the optical guiding means can be produced together with the single lens face as one part by a moulding process. Furthermore, these features may reduce the sensitivity of the optical system to manufacturing tolerances, for example by maintaining a more consistent sensor signal level for different positions of the movable dosage programming component relative to the optical guiding means.

In embodiments of the optical guiding means, the ratio of the sensor-side surface and the encoder-side surface may be about 1.0. Light pipes with such a ratio may contribute to obtaining an optimal signal amplitude generated by the at least one optical sensor when receiving reflection of the emitted radiation through the light pipe.

In yet further embodiments of the optical guiding means, the encoder-side surface may comprise one or more of the following features: it has a textured finish as surface finish, particularly a textured finish according to the D3, D2 or D1 standard of the Society of Plastics Industry SPI. The textured finish may be a finish having a slight roughness or diffusivity such as SPI-D3 or even finer, for example characterized as having features sizes of about 1 μm, particularly nearly equal to the central wavelength of an infrared (IR)—light emitting diode (LED) sensor package, which may be applied as optical sensor. The encoder side surface may have a mirrored finish; the encoder side surface may comprise an antireflection coating. A mirrored finish and an antireflection coating may reflect interfering light and, thus, may prevent that the interfering light enters the light pipe and may reduce the signal-to-noise ratio. The encoder side surface may have an aspherical shaping, allowing to better adapt the encoder side surface to specific reflective surface on the rotary encoder systems. The encoder side surface may also have a spherical shaping, particularly a convex or concave shaping, in order to improve the collection of radiation reflected from the rotary encoder system.

In still further embodiments of the optical guiding means, a side wall of the at least one light pipe may have a mirror finish. This finish may promote total internal reflection (TIR) and efficacy of the light pipe. The mirror finish sidewall may be used in combination with the textured finish of the encoder-side surface, whereby the roughness of the encoder-side surface reduces the amount of TIR at the encoder-side surface as compared to the mirror finish.

In further embodiments of the optical guiding means, a side wall of the at least one light pipe may comprise one or more coatings, wherein the outermost coating may be non-transparent for the guided radiation, or wherein all coatings may be transparent for the guided radiation and an optical refractive index of each of the transparent coatings is smaller than the optical refractive index of the light pipe. This may further improve the light guiding by the at least one light pipe, and may also reduce influences of interfering light for example from outside of the light pipe. Thus, the signal-to-noise-ratio may be improved.

In yet further embodiments of the optical guiding means, the single lens face being formed as collimating optics may have a domed collimating entry face to the internal of the at least one light pipe, wherein the domed collimating entry face comprises a surface shape being different in two orthogonal cross sectional planes. In embodiments, the surface shape the domed collimating entry face may be designed to reduce reflections from the internal surface of the at least one light pipe, parallelise radiation emitted in the internal of the at least one light pipe, and/or to focus reflections of the emitted radiation from the rotary encoder system. This allows to increase the proportion of useful reflections of the emitted radiation present in the light pipe, wherein useful reflections are reflections from the rotary encoder system, particularly a encoding target, while direct reflections from the internal of the light pipe usually do not contribute usefully to determining whether an encoding target is present or absent.

In embodiments of the optical guiding means, the at least one light pipe may have the shape of a conical frustum. This shape is particularly useful for guiding radiation, and is suited for injection moulding.

In another aspect the present disclosure relates to a drug delivery device comprising a movable dosage programming component comprising a rotary encoder system, a sensor arrangement comprising at least one optical sensor configured to detect movement of the movable dosage programming component relative to the sensor arrangement during dosing of a drug by emitting radiation and detecting reflections of the emitted radiation from the rotary encoder system, and optical guiding means as disclosed herein being arranged between the sensor arrangement and the rotary encoder system for guiding radiation emitted by the at least one optical sensor to the rotary encoder system and reflections of the radiation from the rotary encoder system back to the at least one optical sensor. Such a drug delivery device may be for example implemented as injection pen with an elongated body having a distal end comprising a syringe for injecting a selected drug dosage in a patient's body and a proximal end comprising the movable dosage programming component comprising the rotary encoder system. A drug cartridge may be hold in the body, and a mechanism configured to select and deliver a selected dosage of the drug from the cartridge via the syringe may be coupled with the dosage programming component. The sensor arrangement and the optical guiding means may be for example contained in a clip-on module, which can be attached to the distal end of body in order to couple the optical guiding means with the rotary encoder system. For selecting a dosage, a user may rotate the movable dosage programming component via a selection mechanism, and after the selection, the user may press an injection knob to expel the selected drug. The rotation of the movable dosage programming component may be detected via the rotary encoder system, the sensor arrangement and the optical guiding means, and may be processed by an electronics, for example contained in a housing of the sensor arrangement.

In yet another aspect the present disclosure relates to a module configured for application with a drug delivery device and comprising optical guiding means as disclosed herein. The module may be for example implemented as an integrally formed part, particularly an injection moulding part.

In embodiments, the module may further comprise a sensor arrangement comprising at least one optical sensor being configured to detect movement of a movable dosage programming component of the drug delivery device relative to the sensor arrangement during dosing of a drug. Such a module may be for example embodied as add-on module for a disposable drug delivery device such as an injection pen in order to be reusable with another drug delivery device. Thus, the technical complex sensor arrangement must not be cast away with an empty drug delivery device. Data and/or signals generated by the sensor arrangement during detection of a dosage selection may be stored in an internal storage for further processing, or may be transmitted via an interface to electronics, for example via a wireless interface such as a Bluetooth® module, a RFID (Radio Frequency Identification), particularly NFC (Near Field Communication) circuitry, and/or a WLAN (Wireless Local Area Network) module, and/or a wired interface such as an USB (Universal Serial Bus) interface, a SPI (Serial Peripheral Interface).

In embodiments, the module may further comprise electronics with a processor configured to control the at least one optical sensor of the sensor arrangement and to process signals received from the at least one optical sensor of the sensor arrangement to detect a dosage selected with and/or expelled by the drug delivery device. Thus, signal processing may already be performed by the module so that dosage related data are readily available from the module. The dosage related data may be internally stored for downloading to an external device such as a computer or for uploading into a network storage system such as a cloud storage. Such a module may comprise the entire electronics for detecting dosages selected and expelled with a drug delivery device, and may be designed to be used with different drug delivery devices, particularly disposable drug delivery devices.

In specific embodiments, the processor may be configured to control different optical sensors of the sensor arrangement such that radiation is emitted by the different optical sensors in a time-shifted manner such that each optical sensor only receives its own emitted radiation. Emitting radiation in a time shifter manner may comprise activating different optical sensors at different times with time gaps between the activation of different sensors during which emitted radiation can be received. In other words, the emission-reception of different optical sensors may be alternated such that no overlap occurs. Thus, an optical sensor may receive only its own emitted radiation, and not radiation emitted by another optical sensor. This may mitigate the problem of “cross-talk” when two or more optical sensors are activated at the same time or the activation intervals of different sensors overlap, and one optical sensor may receive radiation emitted by another optical sensor.

In further specific embodiments, the processor may be configured to perform the following acts during a calibration phase:

controlling the at least one optical sensor of the sensor arrangement to emit radiation during consecutive time intervals with an increasing duration,
controlling the at least one optical sensor of the sensor arrangement to measure the reflected radiation during the consecutive time intervals,
determining from the measurements of the reflected radiation the time interval among the consecutive time intervals during which a predefined, particularly an optimum amount of reflected radiation was measured, and
storing the duration of the determined time interval as radiation emission duration of the at least one optical sensor during normal usage.

With this proceeding, the at least one optical sensor may be calibrated with regard to the activation of the optical sensor, i.e. the duration of the emission of radiation of the optical sensor may be adjusted such that an under- or over-saturation of the at least one optical sensor may be mitigated, or even avoided. This calibration particularly allows to improving the system efficacy by accounting for manufacturing variations. In some embodiments, a time interval with the highest measured amount of reflected radiation may be selected. In other embodiments, another time interval with a different predefined measured amount of reflected radiation may be selected, for example, where multiple sensors are being used, there may be a requirement to match the amplitude of the sensors closely, and this may be in some embodiments preferred over maximising the amplitude of each sensor.

In embodiments, the module may be configured for attachment to or integration into a drug delivery device comprising a movable dosage programming component comprising a rotary encoder system.

In still another aspect the present disclosure relates to a method for operating a sensor arrangement comprising at least one optical sensor being configured to detect movement of a movable dosage programming component of a drug delivery device relative to the sensor arrangement during dosing of a drug, the sensor arrangement particularly being comprised by a module as disclosed herein, the method comprising the following acts:

controlling the at least one optical sensor of the sensor arrangement to emit radiation during consecutive time intervals with an increasing duration,
controlling the at least one optical sensor of the sensor arrangement to measure the reflected radiation during the consecutive time intervals,
determining from the measurements of the reflected radiation the time interval among the consecutive time intervals during which a predefined, particularly the highest amount of reflected radiation was measured, and
storing the duration of the determined time interval as radiation emission duration of the at least one optical sensor during normal usage of the drug delivery device. In some embodiments, a time interval with the highest measured amount of reflected radiation may be selected. In other embodiments, another time interval with a different predefined measured amount of reflected radiation may be selected, for example, where multiple sensors are being used, there may be a requirement to match the amplitude of the sensors closely, and this may be more important than maximising the amplitude of each sensor. The method may be example implemented by a computer program to be executed by a processor, for example a processor of an electronics of a module as described herein. The method may be particularly implemented by means of a firmware being stored in a memory accessible by the processor. The method may be implemented as a subset of functionality provided by such a firmware.

A yet another aspect of the invention relates to a method for detecting a dosage selected with and/or expelled by a drug delivery device comprising

controlling different optical sensors of a sensor arrangement being particularly comprised by a module as disclosed herein, the at least one, particularly each optical sensor being configured to detect movement of a movable dosage programming component of the drug delivery device relative to the sensor arrangement during dosing of a drug, and
processing signals received from the at least one, particularly each optical sensor of the sensor arrangement to detect a dosage selected with and/or expelled by the drug delivery device, wherein the controlling comprises controlling the different optical sensors of the sensor arrangement such that radiation is emitted by the different optical sensors in a time shifted manner such that each optical sensor only receives its own emitted radiation.

Also this method may be example implemented by a computer program to be executed by a processor, for example a processor of an electronics of a module as described herein. The method may be particularly implemented by means of a firmware being stored in a memory accessible by the processor. The method may be implemented as a subset of functionality provided by such a firmware.

In embodiments, the method may further comprise performing the method for operating a sensor arrangement as described herein in a calibration phase and using the stored duration of the time interval determined in the calibration phase as the radiation emission duration of the at least one optical sensor during normal usage of the drug delivery device, wherein the at least one optical sensor is activated only for the stored duration for emitting radiation. Thus, the activation time of the optical sensor(s) may be better adapted to variations of optical guiding means provided for guiding radiation emitted by the optical sensor(s) to a rotary encoder system and reflections of the emitted radiation.

### BRIEF DESCRIPTION OF THE FIGURES

FIG. 1 shows schematically an example of optical guiding means;

FIGS. 2 to 5 show schematically examples of optical pipes of the optical guiding means;

FIGS. 6 and 7 show an example of a module configured for application with a drug delivery device and comprising optical guiding means;

FIG. 8 shows schematically parts of a module configured for application with a drug delivery device and comprising optical guiding means, a sensor arrangement, and electronics;

FIG. 9 shows an example of a timing diagram with control signals for optical sensors of a sensor arrangement; and

FIG. 10 shows an example course of measurements made during a calibration phase.

### DETAILED DESCRIPTION

In the following, embodiments of the present disclosure will be described with reference to injection devices, particularly an injection device in the form of a pen. The present disclosure is however not limited to such application and may equally well be deployed with other types of drug delivery devices, particularly with another shape than a pen.

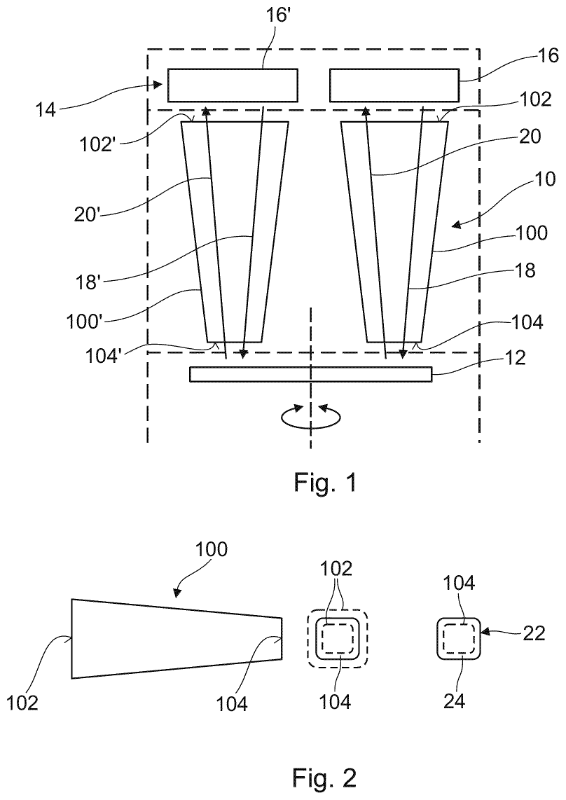

A drug injection pen for application of the optical guiding means and the module as disclosed herein is for example shown in FIG. 1 of WO 2019/101962 A1, which is incorporated herein by reference. The pen may be Sanofi's AllSTAR® injection device where an injection button and grip are combined. The mechanical construction of the AllSTAR® injection device is described in detail in the international patent application WO2014/033195A1, which is incorporated herein by reference. Other injection devices with the same kinematical behaviour of the dial extension and trigger button during dose setting and dose expelling operational mode are known as, for example, the Kwikpen® device marketed by Eli Lilly and the Novopen® device marketed by Novo Nordisk. An application of the general principles to these devices therefore appears straightforward and further explanations will be omitted. However, the general principles of the present disclosure are not limited to that kinematical behaviour. Certain other embodiments may be conceived for application to Sanofi's SoloSTAR® injection device where there are separate injection button and grip components.

The drug injection pen may be equipped with an encoder system. This encoder system may be used to record doses that are delivered from the injection device. The concept of this encoder system is based on a light guidance used to convey the status of an indicator flag to a reflective sensor, which is located physically remote to the flag. FIGS. 21 to 28 of WO 2019/101962 A1 show embodiments of an optical add-on module for an injection device, where the indicator flag is formed by a relative rotation of a number or the dial sleeve and the injection button, the latter of which houses an optical sensor. Such an add-on module may be configured to be added to a suitably configured pen injection device for the purpose of recording doses that are dialed and delivered from the device. This functionality may be of value to a wide variety of device users as a memory aid or to support detailed logging of dose history. The module may be configured to be connectable to a mobile device such as a smartphone or a tablet PC, or similar, to enable the dose history to be downloaded from the module on a periodic basis. However, the concept of the encoder system is also applicable to any device with the indicator flag and sensor separation, for example the injection device 1 of FIG. 1 of WO 2019/101962 A1, wherein the module may be implemented in the dosage knob 12.

FIG. 1 shows optical guiding means 10 configured for application in for example a pen such as the pens described in WO 2019/101962 A1. The optical guiding means 10 are provided to guide radiation 18 from a sensor arrangement 14 to a movable dosage programming component 12, which comprises a rotary encoder system. The component 12 may have a disk-like shape and configured for rotation around its axis. The rotary encoder system may for example several indicator flags such as the flag 1008 shown in FIG. 21 of WO 2019/101962 A1. Reflections 20 of the radiation are guided back from the rotary encoder system to the sensor arrangement 14 via the optical guiding means 10.

The sensor arrangement 14 may comprise at least one optical sensor 16, 16′. The optical sensor 16, 16′ may comprise an IR-LED for emitting IR radiation and a photodiode for detecting reflections of the emitted IR radiation. Also, radiation in another spectrum may be emitted, for example visible light. The emitted radiation may be selected depending on the reflectivity of the rotary encoder system, for example the flags.

The optical guiding means 10 may comprise one or more optical light pipes 100, 100′, which may have the shape of a frustum, particularly a conical frustum. Each light pipe 100, 100′ is arranged between one optical sensor 16, 16′ of the sensor arrangement 14 and the movable dosage programming component 12. The optical sensor 16, 16′ is arranged above a sensor-side surface 102, 102′ of the light pipe 100, 100′ such that nearly the entire radiation emitted by the optical sensor 16, 16′ may enter the light pipe 100, 100′. The rotary encoder system of the component 12 may be arranged below a encoder-side surface 104, 104′ of the light pipe 100, 100′ so that the emitted radiation 18, 18′ is guided by the light pipe 100, 100′ to encoding targets of the rotary encoder system, such as for example the indicator flag 1008 shown in FIG. 21 of WO 2019/101962 A1.

FIG. 2 shows an example of a light pipe 100 in a side view. The light pipe 100 may have a conical cross section owing to the requirement for a taper or “draft” angle to facilitate injection moulding. The average taper angle for any individual light pipe may be characterized by the ratio of the cross-sectional areas of the entry A_{entry}, i.e. the sensor-side surface 102, and the exit Aₑₓᵢₜ, i.e. the encoder-side surface 104. An optimal signal amplitude at the optical sensor emitting radiation into the light pipe 100 and receiving reflections of the emitted radiation through the light pipe 100 may be achieved as A_{entry}/Aₑₓᵢₜ->1. Thus, in order to improve the signal quality, the ratio of the sensor-side surface 102 and the encoder-side surface 104 should be equal to or larger than 1.0 as shown in the middle drawing in FIG. 2. Preferably the ratio should be 1.0 for optimum signal quality. The signal quality can also be improved when the the ratio of the surface 24 (A_{target}) of a discreet encoding target 22 of the rotary encoder system and the encoder-side surface 104 is equal to or larger than 1.0 so that target 22 fully eclipses the encoder-side surface 104, i.e. A_{target}/Aₑₓᵢₜ=>1, as shown in the right drawing in FIG. 2. In this way, the shape of the discreet encoding target 22 and the encoder-side surface 104 may be geometrically nearly identical and nearly orientated in the same manner, but the performance, i.e. the reflectivity of the radiation back into the light may pipe may be agnostic to the exact shape.

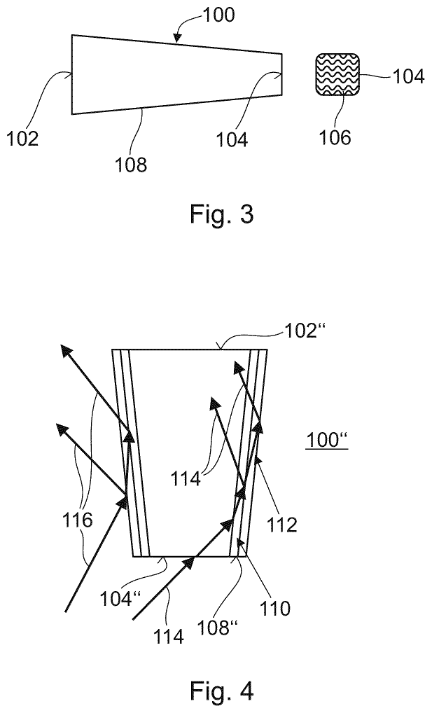

FIG. 3 shows a further example of a light pipe 100 in a side view. To promote TIR and efficacy of the optical system, the conical faces, i.e. the sidewall 108 of the light pipe may be specified as “mirror finish”. Conversely, the exit or encoder-side surface 104 of the light pipe 100 may be specified as having a textured finish 106 with a slight roughness/diffusivity, for example SPI-D3 or even finer.

Particularly the encoder-side surface 104 may have a roughness with an average feature size in the range of the wavelength of the guided radiation, which is emitted by the optical sensor into the light pipe 100. More particularly, the average feature size may be nearly equal to the central wavelength of the radiation guided by the light pipe 100. For example, when IR is guided, the average feature size may be about 1 μm nearly equal to the central wavelength of an IR-LED optical sensor package. The roughness may reduce the amount of TIR at the encoder-side surface 104 as compared to the mirror finish of the sidewall 108, promoting a greater proportion of radiation to interact with the encoding target and be returned to the optical sensor. The encoder-side surface 104 may also have a mirrored finish and/or comprise an antireflection coating for improving reflection of interfering radiation from outside and preventing such radiation from entering the light pipe. Each of these measures may improve the signal-to-noise ratio of an optical sensor arranged at the sensor-side surface of the light pipe.

FIG. 4 shows a further example of a light pipe 100″ with a sidewall 108″ comprising a multi-coating in order to improve the light guidance and shielding against interfering radiation from outside. The sidewall 108″ may comprise a first coating 110 and a second coating 112 covering the first coating 110. A further exterior and nontransparent layer may be provided. Reflected radiation 114 entering the encoder-side surface 104″of the light pipe 100″ with a certain entry angle range may be guided by the coatings 110 and 112. The refraction index n1 of the light pipe's inner core may be larger than the refractive index n2 of the first coating 110, and the refractive index n3 of the second coating 112 is smaller than the refractive index n2: n1>n2>n3. Instead of the second coating 112, the first coating 110 may be also opaque. Thus, the second coating 112 is optional in the example of FIG. 4. The second coating 112 may be also opaque. As can be seen in FIG. 4, the reflected radiation 114 is at least partly guided by the first and second coatings 110 and 112, while “noise” or interfering radiation 116 is also guided at least partly by the coatings 110, 122, but leaves the guiding formed by the coatings 110, 112 and does not enter the inner core of the light guide 100″.

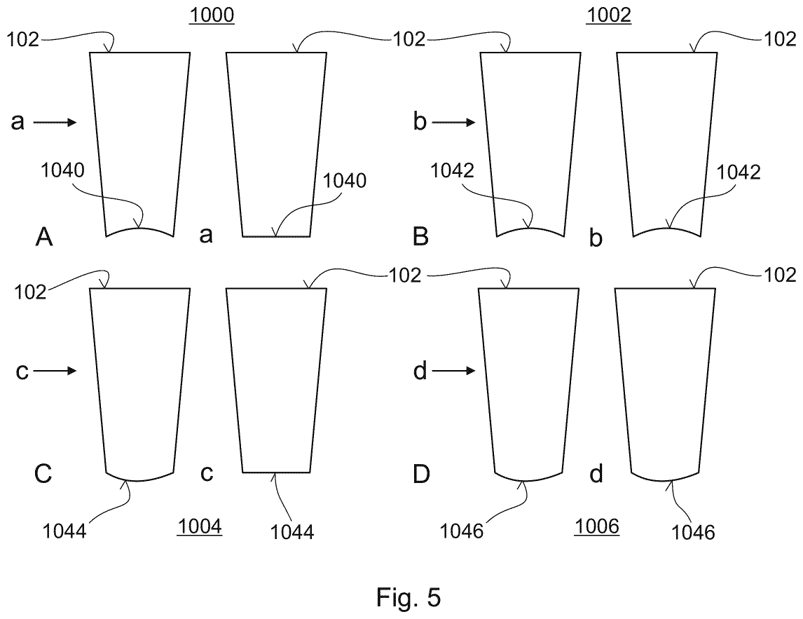

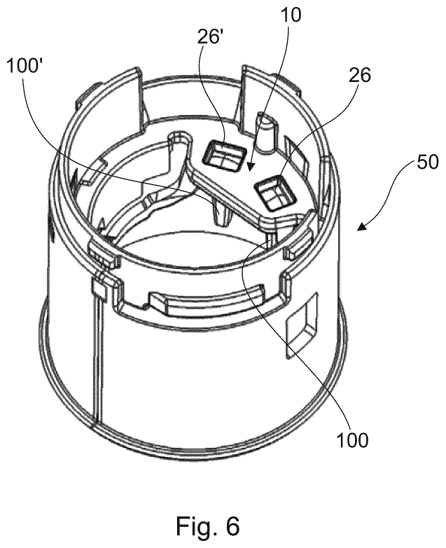

FIG. 5 shows four examples of light pipes 1000, 1002, 1004, 1006 having differently shaped encoder-side surfaces: the light pipes 1000 (drawing A) and 1004 (drawing C) have differently shaped encoder-side surface 1040, 1044 in different radial directions, i.e. an aspherical shaping, as can be seen in the drawings a and c, which shows a side view on the light pipes 1000 and 1004 from the directions a and c. The encoder-side surface 1040 of the light pipe 1000 has in the side view of drawing A a concave shape, while it has a flat shape in the side view of drawing a. The encoder-side surface 1044 of the light pipe 1004 has in the side view of drawing C a convex shape, while it has a flat shape in the side view of drawing c. The light pipes 1002 (drawing B) and 1006 (drawing D) both have spherically shaped encode-side surfaces 1042 and 1046. The encoder-side surface 1042 has concave shape, and the encoder-side surface 1046 has a convex shape. With these different shaping of the encoder-side surfaces 1040, 1042, 10444, 1046, the collecting of reflected radiation for guiding it with the light pipe 1000, 1002, 1004, 1006 back to the optical sensor at the sensor-side surface 102 of the light pipe 1000, 1002, 1004, 1006 may be improved. Also, the encoder-side surface may be better adapted to the reflecting rotary encoder system, for example to differently shaped encoding means such as slanted mirrors. The maximal angle for radiation entering the light pipe may be also better controlled by defining the radius of the spherical shaping of the encoder-side surface (the examples of the light pipes 1002, 1006 shown in drawings B and D). For example, a smaller radius with a more flat spherically shaped encoder-side surface may reduce the angle range, while a larger radius with more bulging may increase the angle range.

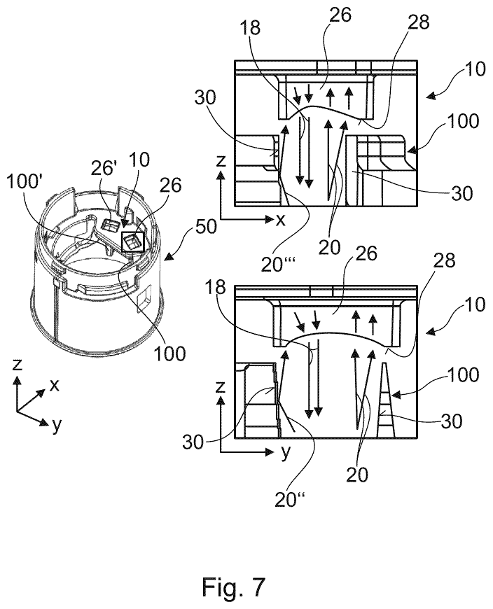

FIG. 6 shows a module 50, for example a moulded part for integration into a drug injection pen or an add-on for attachment to a drug injection pen. The module 50 comprises light pipes 100, 100′, and a single lens face 26, 26′ is being moulded at the sensor-side surface of each light pipe 100, 100′. The module 50 may be integrally formed by moulding. FIG. 7 shows the single lens face 26 in more detail in side view from two different directions corresponding two the two orthogonal planes x-z and y-z. The single lens face 26 comprises a domed collimating entry face 28 to the internal of the light pipe 100. The surface shape of the domed collimating entry face 28 differs in the two orthogonal planes x-z and y-z as can be seen in FIG. 7.

The reflections of radiation, for example IR, emitted into the internal of the light pipe 100 by the optical sensor may be broadly characterized as direct reflections and target reflections. Direct reflections comprises only radiation reflected from the light pipe, i.e. the internal surface of the light pipe. Direct reflections do not contribute usefully to determining whether an encoding target is present or absent. Target reflections comprises radiation, which interacts with the encoding target via the light pipe and therefore contributes directly to the signal produced by the optical sensor from received reflections for decoding whether an encoding target is present or absent. An optimum signal amplitude of the optical sensor may be achieved when the highest proportion of the received reflections comprises target reflections. Then, the amplitude may be dictated by the presence or absence of an encoding target, and not by other reflections in the system. As described in WO 2019/101962 A1, collimating optics may be provided to increase the parallelism of emitted and received radiation, which may increase the reflections from the encoding target among the received radiation reflections.

A refinement as described herein comprises the surface shape of the domed collimating entry face 28 being designed to increase the signal quality of the optical sensor by increasing the proportion of reflections of the radiation emitted into the light pipe 100 by the optical sensor to target reflections. According to the refinement, the domed collimating entry face 28 may have separate, uniquely defined shapes in the x- and y-axes, as shown in FIG. 7. The lens shape is defined to perform the following functions:
significantly reducing reflections 20″, 20′″ from the internal surface 30 of the light pipe 100, leading to lower direct reflections and consequently lower binary ‘0’ values (when not encoding target is below the encoder side surface and no reflections can be received from the missing encoding target);
parallelising or “collimating” radiation 18 emitted in the internal of the light pipe 100 to promote TIR at the particularly conical boundaries of the light pipe 100 and reduce direct reflections at the exit or encoder-side surface of the light pipe, leading to higher binary ‘1’ values and lower binary ‘0’ values, respectively;
collecting and focusing reflections 20 of the emitted radiation from the rotary encoder system, i.e. an encoding target, onto the optical sensor, for example an IR-LED package, leading to higher binary ‘1’ values.

In the same manner as applying diffusivity at the exit or encoder-side surface of the light pipe (FIG. 3), the single lens face at the entry of the light pipe, i.e. the sensor-side surface, may act as an optical “filter”, ensuring a greater amount of useful target reflections may be incident on an optical sensor such as an IR-LED package.

An add-on comprising the module 50 together with electronics may have the ability to detect the binary state of a number sleeve target during a ‘mode shift’, i.e. when a dose button is being depressed from its relaxed state into its 0U position. In this case, the differentiation between a binary ‘0’ and ‘1’ as detected by an optical sensor may be readily obtained for a large, for example ˜0.5 mm separation between the distal or encoder-side surface of the light pipes and the number sleeve, with the compensation undergoing a relative rotation. The incorporation of the integrated single lens faces 26, 26′ may reduce a divergent effect of the aforementioned gap, facilitating easier disambiguation between a ‘0’ and ‘1’ as reported by the optical sensor.

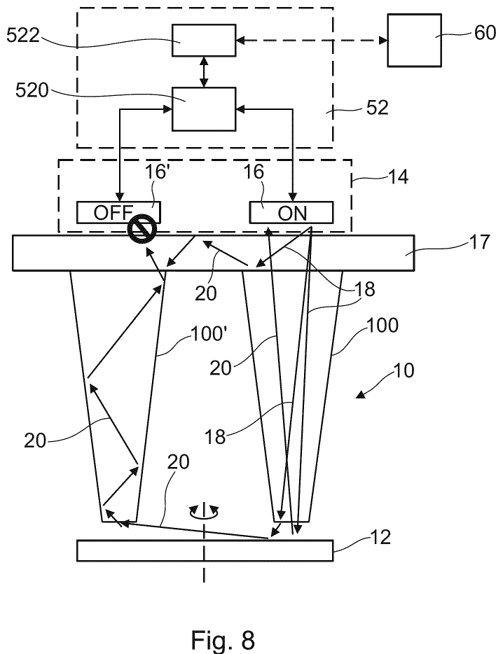

FIG. 8 shows schematically parts of a module, which may be configured for application with a drug delivery device such as an injection pen. The module may for example comprise the module 50 (FIG. 6), which comprises optical guiding means 10 with optical pipes 100, 100′ and single lens faces 26, 26′. The module shown in FIG. 8 may further comprise a sensor arrangement 14 with two optical sensors 16, 16′, and electronics 52 for controlling the optical sensors 16, 16′. The electronics 52 may comprise a processor 520 and communication means 522. The communication means 522 may comprise a wired and/or a wireless interface for communicating for example with an external device 60 such as a computer and/or network storage.

The processor 520 may be configured to control the optical sensors 16, 16′ of the sensor arrangement and to process signals received from the optical sensors 16, 16′ to detect a dosage selected with and/or expelled by the drug delivery device.

While each optical sensor 16, 16′ is provided to interact with a single light pipe 100, 100′, two or even more light pipes 100, 100′ may be present in the optical guiding means 10, which may be implemented by the same plastic component. This may cause that transmitted light from one optical sensor 16 may be received by the other optical sensor 16′ via internal interactions or via the encoding target.

An example of this effect is shown in FIG. 8 by the rays 20 guided through the part 17, which may comprise the above described single face lenses and hold the light pipes 100, 100′. The optical sensor 16 emits radiation 20 directly into the light pipe 100 through a single face lens in part 17, but some rays 20 may via TIR guided within the part 17 and be received by the neighboured optical sensor 16′.

Also, some of the rays 20 may exit the encoder-side surface of the light pipe 100 and reflected by the movable dosage programming component 12 to enter the neighboured light pipe 100′ through the encoder-side surface of this light pipe 100′ and “travel” through the light pipe 100′ to the neighboured optical sensor 100′. Such effects cause a kind of “cross-talk” between both optical sensor 16 and 16′, which may influence the signal generation of the optical sensors. For example, optical sensor 16′ may generate a signal from radiation emitted from optical sensor 16 and vice versa.

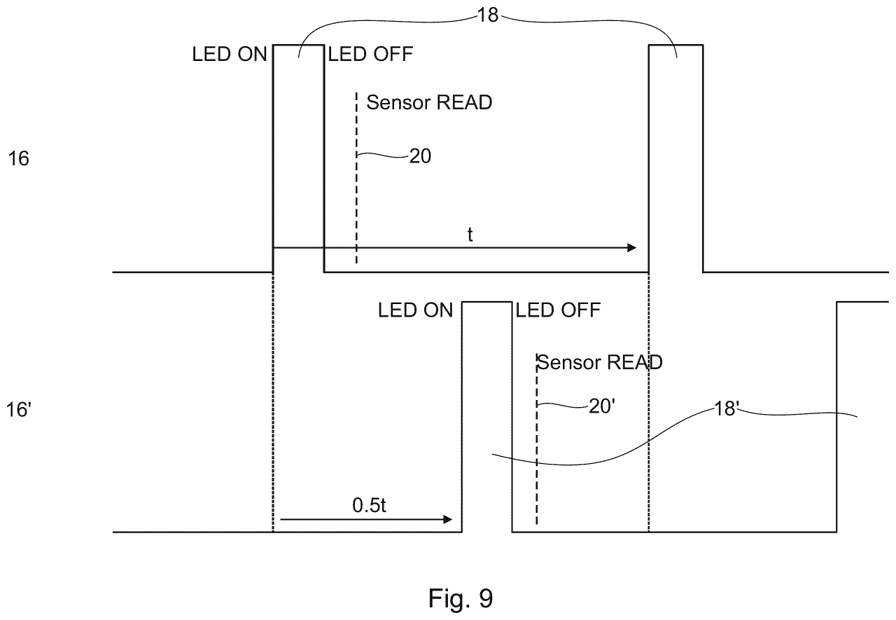

To mitigate this problem, the processor 520 may be configured to alternate or “strobe” the read-write behaviour of the optical sensors 16, 16′ as shown by the example timing diagram of FIG. 9. The diagram shows the activation of the optical sensors 16 and 16′ over the time under control of the processor 520, which is configured to perform a method for detecting a dosage selected with and/or expelled by the drug delivery device, particularly by specific firmware implementing instructions to configure the processor 520 to perform this method. The method performed by the processor 520 comprises controlling the optical sensors 16, 16′, which are configured to detect movement of the movable dosage programming component 12 of the drug delivery device relative to the sensor arrangement 14 during dosing of a drug, and processing signals received from the at least one optical sensor 16, 16′ of the sensor arrangement 14 to detect a dosage selected with and/or expelled by the drug delivery device. The controlling comprises controlling the optical sensors 16, 16′ such that radiation is emitted by the different optical sensors 16, 16′ in a time shifted manner such that each optical sensor 16, 16′ only receives its own emitted radiation.

The method performed by the processor 520 is now explained in detail with particular reference to the timing diagram of FIG. 9. The activation phases 18, 18′ of the sensors 16, 16′ are also designated by “LED ON” and “LED OFF”. The activation of the different sensors 16 and 16′ is time-shifted, i.e. only one of the sensors 16, 16′ emits radiation in a certain time interval, as can be seen in FIG. 9. The activations 18, 18′ of each of each optical sensor 16, 16′ may be periodically with a time interval t. The alternation of the activation of the sensors 16 and 16′ may be time-shifted by 0.5*t, i.e. a half of the time interval t. The duration of an activation 18, 18′ may be for example 0.1*t or 0.2*t so that 0.4*t or 0.3*t remain in the half of a time interval t for activate the read or reception mode of the optical sensor 16, 16′ to receive the reflections 20, 20′ of the previously emitted radiation 18, 18′. In the diagram from FIG. 9, first the optical sensor 16 emits radiation 18, and thereafter with a short delay of for example 0.1*t the processor 502 switches the optical sensor 16 into a read or reception mode to receive the reflections 20 of the previously emitted radiation 18. The switching in the read or reception mode may be time limited to a relatively short duration, which is selected such that enough reflections 20 may be received to generate a signal with a large enough amplitude for decoding by the processor 502. When the read or reception mode is terminated by the processor 502, the optical sensor 16′ is activated by the processor 502 to emit radiation 18′, and thereafter with a short delay of for example 0.1*t the processor 502 switches the optical sensor 16′ into a read or reception mode to receive the reflections 20′ of the previously emitted radiation 18. The processor 502 may then proceed with this alternating activation of both sensor 16, 16′. The above timings may be specified according to the electronic response of each optical sensor 16, 16′ and the expected transition frequency of the rotary encoder system, particularly encoding flags of such an encoder system. Consequently, at any moment in time, only the returned or reflected radiation associated with one specific light pipe and a target beneath the light pipe and associated optical sensor being interrogated may contribute to the signal generated by this sensor. Radiation transmitted through one pipe and reflected into another pipe associated with another optical sensor does not contribute to the signal generation since the other optical sensor is at that time not activated due to the time-shifted activation.

With this time-shifted activation of different optical sensors, it may be ensured that each sensor receives only its own emitted radiation, and not radiation emitted from another sensor. Thus, “cross-talk” between neighboured optical pipes and their assigned optical sensors may be mitigated if not avoided.

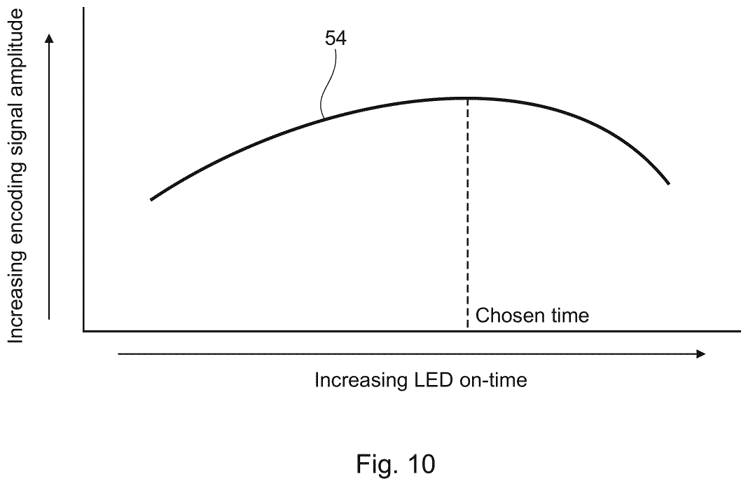

The processor 520 may be also configured to perform a method for calibrating the sensor arrangement 14 with the optical sensors 16, 16′ by performing certain acts during a calibration phase as will be described in the following in more detail with reference to FIGS. 9 and 10. As shown in FIG. 9, the time between “LED-ON” and “LED-OFF” may be referred to the LED-ON or sensor activation time. If the LED-ON time is too small, insufficient reflected radiation may be collected or received by an optical sensor causing an under-saturation of for example a photodiode of the optical sensor. The result of under-saturation may be a lower-than-optimum binary ‘1’ signal value and hence a lower signal amplitude, which may make further processing and particularly encoder detection by the processor 520 more difficult and unreliable. Similarly, if the LED-ON time is too large, the optical sensor may receive too much reflections of radiation and may become saturated, i.e. the binary ‘1’ signal may reach a maximum. Concurrently, since there is a greater amount of received radiation in the system, the binary ‘0’ signal may also be forced higher due to a finite proportion of direct reflections. It follows that if the binary ‘0’ signal may rise, but the binary ‘1’ signal cannot due to the saturation, and then the resultant amplitude would decrease.

The processor 520 may be configured to perform a calibration phase for finding the optimum LED-ON time with regard to saturation. The optimum LED-ON time would deliver the highest signal amplitude. During the calibration phase, the processor 520 may be configured to perform vary the LED-ON time between two extreme values and to measure the reflected radiation over the variation. The LED-ON time with the measured highest amount of reflected radiation may be determined as the optimum LED-ON time since it may deliver the highest signal amplitude. The following acts performed by the processor 520 during the calibration phase may be as follows:
controlling the optical sensor 16, 16′ of the sensor arrangement 14 to emit radiation during consecutive time intervals with an increasing duration,
controlling the optical sensor 16, 16′ of the sensor arrangement 14 to measure the reflected radiation during the consecutive time intervals (an example course of measurements 54 made during a calibration phase is shown in FIG. 10),
determining from the measurements 54 of the reflected radiation the time interval among the consecutive time intervals during which the highest amount of reflected radiation was measured, and
storing the duration of the determined time interval as radiation emission duration of the at least one optical sensor 16, 16′ during normal usage.

It should be noted that the measured reflected radiation may comprise the amplitude of the output signal of the optical sensor 16, 16′, which may be processed by the processor 520 for decoding. The above calibration phase may also account for manufacturing variations of the optical guiding means, for example of the moulded module 50 from FIG. 6, particularly for geometric tolerances and/or material property differences, and of the sensor packages.

By calibrating out the aforementioned variations, the signal response may be normalised such that a decoding algorithm particularly executed by the processor 520 may be made to operate on waveforms with similar characteristics, allowing for further particularly software-based optimisations that may increase reliability and reduce power consumption.

During normal usage, particularly when the processor 520 is activated to detect a dosage selected with and/or expelled by the drug delivery device, the stored duration of the determined time interval, which was determined in a calibration phase as the radiation emission duration of the optical sensors 16, 16′ during normal usage, can then be used by the processor 520 for controlling the radiation emission by the optical sensors 16, 16′. The calibration phase as describe above may be also automatically performed before, during and/or after a normal usage. Additionally or alternatively, the calibration phase may be initiated by a user manually, for example by pressing a button for a certain time to configure the processor 520 to execute the acts of the calibration phase.

The above described embodiments comprise
the implementation of geometric relationships and the application of surface finishes to light pipes of optical guiding means used to convey radiation such as IR between a geometrically separated optical sensor and a reflective target,
the definition of a bi-directional lens at the junction between an optical sensor and the entry- or sensor-side-surface of a light pipe of optical guiding means, which may in turn increase the proportion of useful reflected radiation in the system,
the use of an alternating illumination or “strobing” methodology to reduce “cross-talk” between optical sensors within an optical system applicable for detecting dosage selection by a drug delivery device,
the use of a calibration step to normalize the performance of an optical system applicable for detecting dosage selection by a drug delivery device by reducing effects of manufacturing variations.

The benefit of the above described embodiments is to create a signal waveform—generated by the rotation of an encoding target between target present—binary ‘1’—and target absent—binary ‘0’—which may have large swings and common rail voltages. These characteristics may result in a signal that can be more easily and reliably encoded.

The benefit may be particularly important for a system such as an optical decoder, where the power consumption of an optical sensor, for example an IR-LED may account for a significant proportion of the total power consumption.

By incorporating the methodologies and techniques as herein described, an optical sensor, for example an IR-LED may be configured to have a lower operating current and receive a more consistent signal that is agnostic to geometric variations in the optical guiding means.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively, or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism.

Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N- tetradecanoyl)-des(B30) human insulin (insulin detemir, Levemir®, B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl- ThrB29LysB30 human insulin;

B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C, CM-3, GLP-1 Eligen, ORMD-0901, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, TT-401, BHM-034. MOD-6030, CAM-2036, DA-15864, ARI-2651, ARI-2255, Exenatide-XTEN and Glucagon-Xten.

An examples of an oligonucleotide is, for example: mipomersen sodium (Kynamro®), a cholesterol-reducing antisense therapeutic for the treatment of familial hypercholesterolemia.

Examples of DPP4 inhibitors are Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

1-17. (canceled)

18. An optical guiding device configured for application with a drug delivery device that has (i) a movable dosage programming component comprising a rotary encoder system and (ii) a sensor arrangement comprising at least one optical sensor, the at least one optical sensor configured to detect movement of the movable dosage programming component relative to the sensor arrangement during dosing of a drug by emitting radiation and detecting at least one reflection of the emitted radiation from the rotary encoder system,
wherein the optical guiding device is configured to guide the radiation and the at least one reflection of the emitted radiation and comprises at least one light pipe having the shape of a frustum with a sensor-side surface and an encoder-side surface, and
wherein the at least one light pipe comprises one or more of the following features:
a ratio of the sensor-side surface and the encoder-side surface being equal to or larger than about 1.0;
a ratio of a surface of a discreet encoding target of the rotary encoder system and the encoder-side surface being equal to or larger than 1.0;
the encoder-side surface having a roughness with an average feature size in a range of a wavelength of the radiation emitted by the at least one optical sensor; or
the sensor-side surface forms a single lens face being molded as part of the optical guiding device, wherein the single lens face is formed as collimating optics.

19. The optical guiding device of claim 18, wherein the encoder-side surface comprises one or more of the following features:
the encoder-side surface has a textured finish as surface finish;
the encoder-side surface has a mirrored finish;
the encoder-side surface comprises an antireflection coating;
the encoder-side surface has an aspherical shaping; or
the encoder-side surface has a spherical shaping.

20. The optical guiding device of claim 19, wherein the textured finish is a finish having a slight roughness or diffusivity.

21. The optical guiding device of claim 19, wherein the textured finish is a finish according to the D3, D2 or D1 standard of the Society of Plastics Industry (SPI).

22. The optical guiding device of claim 18, wherein a side wall of the at least one light pipe has a mirror finish.

23. The optical guiding device of claim 18, wherein a side wall of the at least one light pipe comprises one or more coatings, wherein an outermost coating is non-transparent for the guided radiation, or wherein all coatings are transparent for the guided radiation and an optical refractive index of each of the transparent coatings is smaller than an optical refractive index of the light pipe.

24. The optical guiding device of claim 18, wherein the single lens face being formed as collimating optics has a domed collimating entry face internal to the at least one light pipe, wherein the domed collimating entry face comprises a surface shape different in two orthogonal cross sectional planes.

25. The optical guiding device of claim 24, wherein the surface shape of the domed collimating entry face is designed to reduce reflections from the internal surface of the at least one light pipe, parallelize radiation emitted in the internal of the at least one light pipe, and/or to focus reflections of the emitted radiation from the rotary encoder system.

26. The optical guiding device of claim 18, wherein the at least one light pipe has the shape of a conical frustum.

27. A drug delivery device comprising
a movable dosage programming component comprising a rotary encoder system;
a sensor arrangement comprising at least one optical sensor configured to detect movement of the movable dosage programming component relative to the sensor arrangement during dosing of a drug by emitting radiation and detecting at least one reflection of the emitted radiation from the rotary encoder system; and
an optical guiding device arranged between the sensor arrangement and the rotary encoder system for guiding radiation emitted by the at least one optical sensor to the rotary encoder system and the at least one reflection of the radiation from the rotary encoder system back to the at least one optical sensor,
wherein the optical guiding device comprises at least one light pipe having the shape of a frustum with a sensor-side surface and an encoder-side surface, and
wherein the at least one light pipe comprises one or more of the following features:
a ratio of the sensor-side surface and the encoder-side surface being equal to or larger than 1.0;
a ratio of a surface of a discreet encoding target of the rotary encoder system and the encoder-side surface being equal to or larger than 1.0;
the encoder-side surface having a roughness with an average feature size in a range of a wavelength of the radiation emitted by the at least one optical sensor; or
the sensor-side surface forms a single lens face being molded as part of the optical guiding device, wherein the single lens face is formed as collimating optics.

28. A module configured for application with a drug delivery device and comprising an optical guiding device,
wherein the optical guiding device comprises at least one light pipe having the shape of a frustum with a sensor-side surface and an encoder-side surface, and
wherein the at least one light pipe comprises one or more of the following features:
a ratio of the sensor-side surface and the encoder-side surface being equal to or larger than 1.0;
a ratio of the surface of a discreet encoding target of a rotary encoder system and the encoder-side surface being equal to or larger than 1.0;
the encoder-side surface having a roughness with an average feature size in a range of a wavelength of the radiation emitted by the at least one optical sensor; or
the sensor-side surface forms a single lens face being molded as part of the optical guiding device, wherein the single lens face is formed as collimating optics.

29. The module of claim 28, further comprising a sensor arrangement comprising at least one optical sensor being configured to detect movement of a movable dosage programming component of the drug delivery device relative to the sensor arrangement during dosing of a drug.

30. The module of claim 29, further comprising electronics with a processor configured to control the at least one optical sensor of the sensor arrangement and to process signals received from the at least one optical sensor of the sensor arrangement to detect a dosage selected with and/or expelled by the drug delivery device.

31. The module of claim 30, wherein the processor is configured to control different optical sensors of the sensor arrangement such that radiation is emitted by the different optical sensors in a time shifted manner such that each optical sensor only receives its own emitted radiation.

32. The module of claim 30, wherein the processor is configured to perform the following operations during a calibration phase:
controlling the at least one optical sensor of the sensor arrangement to emit radiation during consecutive time intervals with an increasing duration,
controlling the at least one optical sensor of the sensor arrangement to measure the reflected radiation during the consecutive time intervals,
determining from the measurements of the reflected radiation the time interval among the consecutive time intervals during which an optimum amount of reflected radiation was measured, and
storing the duration of the determined time interval as radiation emission duration of the at least one optical sensor during normal usage.

33. The module of claim 28, being configured for attachment to or integration into a drug delivery device comprising a movable dosage programming component comprising the rotary encoder system.

34. A method for operating a sensor arrangement that comprises at least one optical sensor being configured to detect movement of a movable dosage programming component of a drug delivery device relative to the sensor arrangement during dosing of a drug, the method comprising:
controlling the at least one optical sensor of the sensor arrangement to emit radiation during consecutive time intervals with an increasing duration,
controlling the at least one optical sensor of the sensor arrangement to measure the reflected radiation during the consecutive time intervals,
determining from the measurements of the reflected radiation the time interval among the consecutive time intervals during which an optimum amount of reflected radiation was measured, and
storing the duration of the determined time interval as radiation emission duration of the at least one optical sensor during normal usage of the drug delivery device,
wherein the sensor arrangement is included in a module configured for application with the drug delivery device and comprising an optical guiding device, wherein the optical guiding device comprises at least one light pipe having the shape of a frustum with a sensor-side surface and an encoder-side surface, and
wherein the at least one light pipe comprises one or more of the following features:
a ratio of the sensor-side surface and the encoder-side surface being equal to or larger than 1.0;
a ratio of the surface of a discreet encoding target of a rotary encoder system and the encoder-side surface being equal to or larger than 1.0;
the encoder-side surface having a roughness with an average feature size in a range of a wavelength of the radiation emitted by the at least one optical sensor; or
the sensor-side surface forms a single lens face being molded as part of the optical guiding device, wherein the single lens face is formed as collimating optics.

35. The method of claim 34, further comprising: using the stored duration of the time interval determined in the calibration phase as the radiation emission duration of the at least one optical sensor during normal usage of the drug delivery device, wherein the at least one optical sensor is activated only for the stored duration for emitting radiation.

36. A method for detecting a dosage selected with and/or expelled by a drug delivery device, the method comprising:
controlling different optical sensors of a sensor arrangement, at least one optical sensor being configured to detect movement of a movable dosage programming component of the drug delivery device relative to the sensor arrangement during dosing of a drug; and
processing signals received from the at least one optical sensor of the sensor arrangement to detect a dosage selected with and/or expelled by the drug delivery device, wherein the controlling comprises controlling the different optical sensors of the sensor arrangement such that radiation is emitted by the different optical sensors in a time shifted manner such that each optical sensor only receives its own emitted radiation,
wherein the sensor arrangement is included in a module configured for application with the drug delivery device and comprising an optical guiding device, wherein the optical guiding device comprises at least one light pipe having the shape of a frustum with a sensor-side surface and an encoder-side surface, and
wherein the at least one light pipe comprises one or more of the following features:
a ratio of the sensor-side surface and the encoder-side surface being equal to or larger than 1.0;
a ratio of the surface of a discreet encoding target of a rotary encoder system and the encoder-side surface being equal to or larger than 1.0;
the encoder-side surface having a roughness with an average feature size in a range of a wavelength of the radiation emitted by the at least one optical sensor; or
the sensor-side surface forms a single lens face being molded as part of the optical guiding device, wherein the single lens face is formed as collimating optics.

37. The method of claim 36, wherein controlling the different optical sensors of the sensor arrangement such that radiation is emitted by the different optical sensors in a time shifted manner comprises:
activating the different optical sensors at different times with time gaps between the activation of different optical sensors such that no overlaps occur.
